(19) United States
(12) Patent Application Publication
(10) Pub. No.: US 2016/0158211 A1
(43) Pub. Date: Jun. 9, 2016

(54) Dosage and Use of an A2A Antagonist

(71) Applicant: H. Lundbeck A/S, Valby (DK)

(72) Inventors: Lone Frydelund Larsen, Kgs. Lyngby (DK); Johan Areberg, Limhamn (SE); Nathalie Breysse, Copenhagen (DK); Gamini Chandrasena, Monroe, NY (US)

(73) Assignee: H. Lundbeck A/S, Valby (DK)

(21) Appl. No.: 14/955,148

(22) Filed: Dec. 1, 2015

(30) Foreign Application Priority Data
Dec. 3, 2014 (DK) PA201400704

(51) Int. Cl. A61K 31/4439; A61K 9/20

(52) U.S. Cl. CPC A61K 31/4439; A61K 9/2004

(57) ABSTRACT

The present invention relates to specific dosages of an A_{2A} antagonist for use as a medicament and in particular useful for the treatment of Parkinson Disease and Attention Deficit/Hyperactivity Disorder.

### FIELD OF THE INVENTION

The present invention encompasses i.a. two parameters: Use of the A_{2A} receptor antagonist concept for the treatment of attention deficits hyperactivity disorder and to specific dosages of said A_{2A} antagonist for the treatment of Parkinson's Disease and Attention Deficit/Hyperactivity Disorder.

### BACKGROUND OF THE INVENTION

A_{2A} receptor antagonists have attracted considerable interest as potential target for various CNS disorders taping into the fronto-striatal circuitry. Clinical development of A_{2A} antagonists (istradefylline, preladenant and tozadenant) have focused on the treatment of Parkinson's Disease (PD).

Adenosine A_{2A} receptor antagonists represent a new way forward in the treatment of PD via a non-dopaminergic mechanism. In the context of PD, A_{2A} antagonists improve motor function without worsening dyskinesia in pre-clinical models. Clinical data supports the potential for A_{2A} antagonism in the motor component of PD. Furthermore, A_{2A} antagonists have been shown pre-clinically to ameliorate cognitive dysfunction, anxiety and depression thereby highlighting the potential for efficacy in the neuropsychiatric components of PD. To date, the only A_{2A} receptor antagonist on the market for the treatment of PD is istradefylline with starting dose of 20-40 mg daily.

Compound 504 is a new adenosine A_{2A} receptor antagonist under development and has been disclosed in patent applications WO2005/063743 (compound 504) and WO2010/126082. The present invention discloses i.a. 1) a new surprisingly low therapeutic dosage of compound 504, 2) extended half-life compared to other A_{2A} antagonists, therewith supporting longer duration of effect and its application in 3) CNS disorder with particular focus on PD and Attention Deficit/Hyperactivity Disorder (ADHD).

ADHD is a chronic and highly heritable developmental disorder characterised by symptoms of attentional impairment, impulsive actions, hyperactivity as wells as cognitive dysfunctions. ADHD afflicts 5% to 10% of school aged-children and up to 5% of adults worldwide. Although the aetiology of ADHD remains unknown, there is emerging evidence for delayed brain maturation and altered connectivity within cortico-cortical as well as cortico-subcortical circuitries in ADHD. Large body of literature denotes hypoactivation in 1) fronto-striatal network and 2) fronto-temporal network (Rubia K et al, Am J Psychiatry. 1999 June; 156(6):891-6, Rubia K et al, Am J Psychiatry. 2005 June; 162(6):1067-75, and Rubia K, et al Neuropsychopharmacology. 2011 July; 36(8):1575-86; Epstein J N, et al, J Child Psychol Psychiatry. 2007 September; 48(9):899-913) in ADHD. The fronto-striatal hypoactivation in ADHD has been linked to the motor and interference inhibition (Aron A R, and Poldrack R A, J Neurosci. 2006 Mar. 1; 26(9):2424-33.; Rubia K, et al., J Abnorm Child Psychol. 2003 June; 31(3):301-13) as well as sustained and selective attentional processes (Smith A B, et al Am J Psychiatry. 2006 June; 163(6):1044-51).

Treatment options for ADHD are pharmacotherapy (stimulants and non-stimulants), psychoeducation and cognitive behavioural therapy. First and second line psychopharmacological treatments for ADHD are extended release stimulants (methylphenidate and amphetamine based). Stimulants have been consistently shown to improve core symptom deficits in ADHD in children as well as adults. Non-stimulants, in particular atomoxetine, have also been found to be efficacious in the treatment of core domain deficits within ADHD. The mode of action of ADHD medication remains to be fully elucidated but to date evident suggests that the benefits observed with both the stimulant and non-stimulant medications are tightly linked to increased dopamine (DA) and norepinephrine (NE) neurotransmission particularly in the pre-frontal cortex. Furthermore, at the neural network level, methylphenidate, improves sustained attention and impulsivity and concomitantly normalizes fronto-striatal networks while not altering medial frontal and temporal dysfunction (Rubia K, et al Neuropsychopharmacology. 2011 July; 36(8):1575-86, Cubillo A, et al., J Psychiatr Res. 2010 July; 44(10):629-39, and Cubillo A, et al., Cereb Cortex. 2014 January; 24(1):174-85). In summary, the ADHD literature to date suggests that while current medication is clinically efficacious in treating the core domains of ADHD via improved fronto-striatal connectivity and increased prefrontal DA and NE, a number of unmet needs including cognitive dysfunction associated with the disease remain to be fully addressed (Gualtieri C T, and Johnson L G. J Atten Disord. 2008 January; 11(4):459-69, Bidwell L C, et al., Pharmacol Biochem Behay. 2011 August; 99(2):262-7, and Swanson J, et al., Neuropsychopharmacology. 2011 January; 36(1):207-26).

Although ADHD patients respond favourably to stimulants, they carry dependence potential, stigma and are marked by black box labels (risk of abuse, sudden death, and suicidality).

Stimulants are very efficacious in the treatment of ADHD, however, the safety concerns with current medication (see above) highlights the considerable interest in identifying novel safe alternatives. Lastly, while very efficacious and exerting an effect size of close to 1, stimulants are active for 3-16 hours depending on formulation, thereby alleviating ADHD symptomatology during the day. ADHD however is a disorder which afflicts work/school as well as social life and thus there is a need for a 24 hour medication to support morning and evening social and academic functioning for the purpose of improved quality of life for patients.

No adenosine A_{2A} receptor antagonists are currently on the market for the treatment of ADHD. Recently, V-81444 (disclosed in WO2002055082, compound 14) was tested in a phIb/II proof of concept study using a twice daily dosage regiment of 100 mg. The need for a twice-daily dosing regimen is supported by the finding of V-81444's 5-7 hour half-life (Abstracts/Journal of the Neurological Sciences 333 (2013), Abstract No: 951) and a Ki of ˜2 nM.

The present invention highlights compound 504 as a low dose, once-daily ADHD medication with novel Mode of Action (MoA). The novel MoA highlights the potential for efficacy within the ADHD core domains in addition to the cognitive and emotional dysfunction in ADHD. Furthermore, while increasing efficacy, 504 may result in decreased side effects due to the A_{2A} MoA and low dosage need. Stimulant medications, while being first and second line of treatment are linked to poor adherence in particular in the adult population due to lack of predictability in dose selection. The 504 compound would be used in a low and narrow dose range thereby supporting adherence to the medication. Lastly, the once daily dosing may support improved morning and evening functioning compared to stimulants. The invention thus provides a safe and effective novel ADHD medication.

### SUMMARY OF THE INVENTION

The present invention relates to compound 504 as presented by the below formula

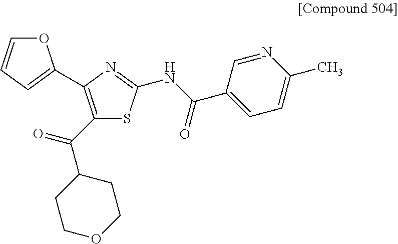 [SMILES: Cc1ccc(C(=O)Nc2nc(-c3ccco3)c(C(=O)C3CCOCC3)s2)cn1]

and pharmaceutical acceptable salts thereof.

for use as a medicament at dosage of 2-3 mg, such as a once daily dosage of 2-3 mg.

In one aspect the invention relates to compound 504 for use in treating PD or ADHD.

Furthermore, the invention relates to a method of treatment of patients with ADHD and PD using compound 504 in a dosage of 2-3 mg, such as a 2 or 3 mg daily dosage.

### BRIEF DESCRIPTION OF DRAWINGS

FIG. 1: Pharmacokinetic and Pharmacodynamic Results

Receptor occupancy in the rat putamen versus the compound 504 plasma concentration at the time of the brain slice autoradiography scan estimated using the Eₘₐₓ method. X-axis show the receptor occupancy (%)

FIG. 2: Predicted therapeutically effective EC₅₀₋₈₀ of compound 504 plasma concentration range in PD patients based on rat A_{2A} occupancy and MPTP-treated marmoset disability reversal scores. X-axis shows plasma concentration (ng/ml), Y-axis (left) shoes A2A % RO (Receptor Occupancy), Y-axis (right) shows disability reversal. ▴A_{2A} % RO,—non-linear fit receptor occupancy; ▪ Disability reversal,—non-linear fit disability reversal.

FIG. 3: Receptor occupancy in the putamen versus the compound 504 plasma concentration at the time of the PET scan estimated using the Eₘₐₓ. X-axis shows CPET (ng/ml), Y-axis Occupancy (%). 5 mg, [symbol] 3 mg, [symbol] 2 mg, □ 1 mg ◯ 0.5 mg,—curve fit.

### DETAILED DESCRIPTION OF THE INVENTION

By the term “compound 504” is intended to mean a compound according to the following formula

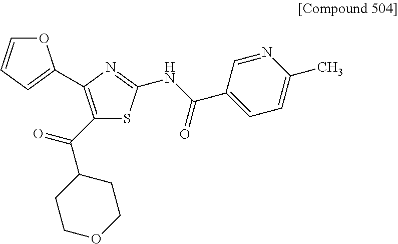 [SMILES: Cc1ccc(C(=O)Nc2nc(-c3ccco3)c(C(=O)C3CCOCC3)s2)cn1]

and pharmaceutical acceptable salts thereof.

In the brain A_{2A} receptors are abundantly expressed in the striatum, nucleus accumbens, globus pallidus, and the olfactory bulb, with low expression levels in other brain regions where they play an important role in regulating synaptic transmission of glutamate and dopamine In the dorsal striatum, A_{2A} receptors are in tight physical and functional interaction with the dopamine neurotransmitter system. A_{2A} receptors are co-localised with D2 receptors on the GABA-ergic striatopallidal medium spiny neurons, where there is evidence for functional antagonism between A_{2A} and D2 receptors; A_{2A} antagonism can lead to similar responses as D2 agonism within the indirect output pathway. Thus, A_{2A} receptor antagonists enhance dopaminergic transmission in the cortex and basal ganglia.

Medications with dopaminergic and noradrenergic activity seem to reduce ADHD symptoms by blocking dopamine and norepinephrine reuptake, and structural and functional imaging studies suggest that dysfunction in the fronto-subcortical pathways, as well as imbalances in the dopaminergic and noradrenergic systems, contribute to the pathophysiology of ADHD. The influence of A_{2A} antagonists on dopamine release in the central nervous system, and the results presented herein suggest that compound 504 will be effective in treating ADHD symptoms. Indeed, and in addition to clinical observations, the putative interest of A_{2A} receptors in ADHD has been emphasised by preclinical studies demonstrating beneficial effects of A_{2A} antagonists on attention deficits measured both in spontaneous hypertensive rats (Takahashi et al., 2008, Front Biosci 13:2614-2632; Pires et al., 2009, Behav Pharm March, Vol 20, Issue 2: 134-145) but also in rats cognitive dysfunctions measured in a test of selective attention, the 5-choice serial reaction time task (Higgins et al Behav Brain Res (2007); 185 (1): 32-42). Importantly, the deficits in attention were reversed with methylphenidate treatment at therapeutically relevant dose range for ADHD.

In vitro, compound 504 possesses high affinity for human, marmoset, rodent, and dog A_{2A} receptor (0.1<Ki [nmol]<0.3) as shown in the table below. Compound 504 is a very selective A_{2A} receptor antagonist, not only relative to other adenosine receptor subtypes, but also to a broad range of CNS targets, including receptors, ion channels and transporters. Less than 50% inhibition of binding at 10 μM was found for any of the 57 counter-screen targets. The selectivity for A_{2A} receptor over A1 receptors is 872 times in humans, 2700 times in marmosets, 500 times in rats, 97 times in mice, and 1590 times in dogs. Little or no affinity was found for subtypes of various other receptors (including DA, 5 hydroxytryptophan [5-HT], acetylcholine [Ach], GABA, glutamate [Glu], opiates, cannabinoid), ion channels (calcium, potassium) and transporters (adenosine, monoamine, serotonin, dopamine, and norepinephrine).

Compound 504
Ki (nmol/L)

| | A₁ | A_{2A} | A_{2B} | A₃ |
|---|---|---|---|---|
| Human | 103 ± 18.4 | 0.118 ± 0.0023 | 33.8 ± 5.91 | 428 ± 72.5 |
| Marmoset | 305 ± 6.6 | 0.113 ± 0.0046 | ND | ND |
| Dog | 450 ± 51.1 | 0.283 ± 0.0269 | ND | ND |
| Rat | 50.7 ± 3.18 | 0.100 ± 0.0072 | ND | ND |
| Mouse | 31.1 ± 1.09 | 0.322 ± 0.0114 | ND | ND |

ND = not determined

As shown in Example 1 of the present invention the pharmacokinetic (PK)/pharmacodynamic (PD) relationship of compound 504 was evaluated using rat striatal A_{2A} occupancy (Example 1) as well as the disability reversal score in the more clinically relevant MPTP-lesion marmosets (Example 2).

An administration of an A_{2A} antagonist which will give receptor occupancy of about 80% can in most instances be assumed enough to induce a clinical relevant response (Mihrara et al. J Nucl Med (2008) 49:1183-1188 and Brooks et al. Synapse (2008) 62:671-681).

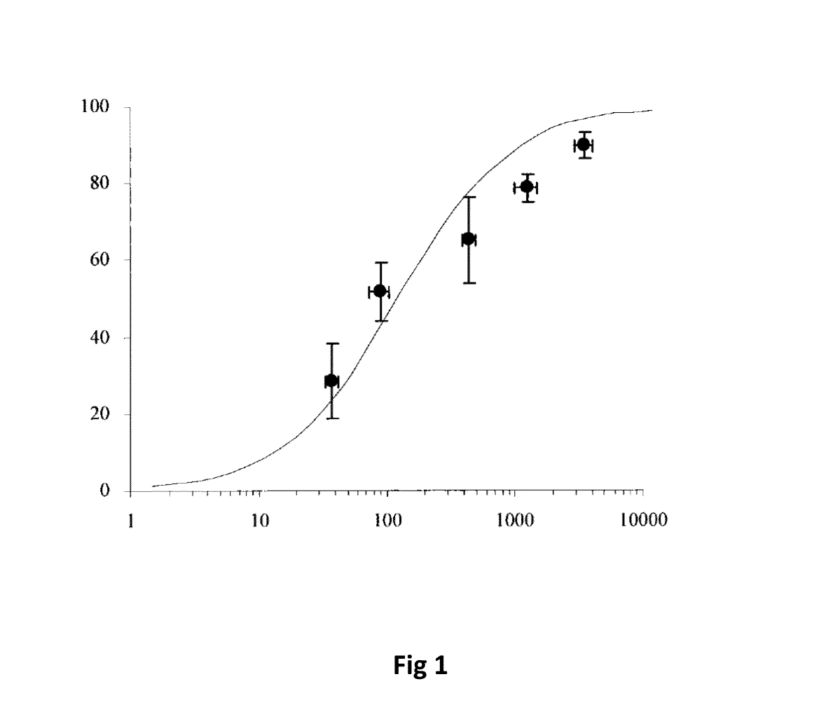

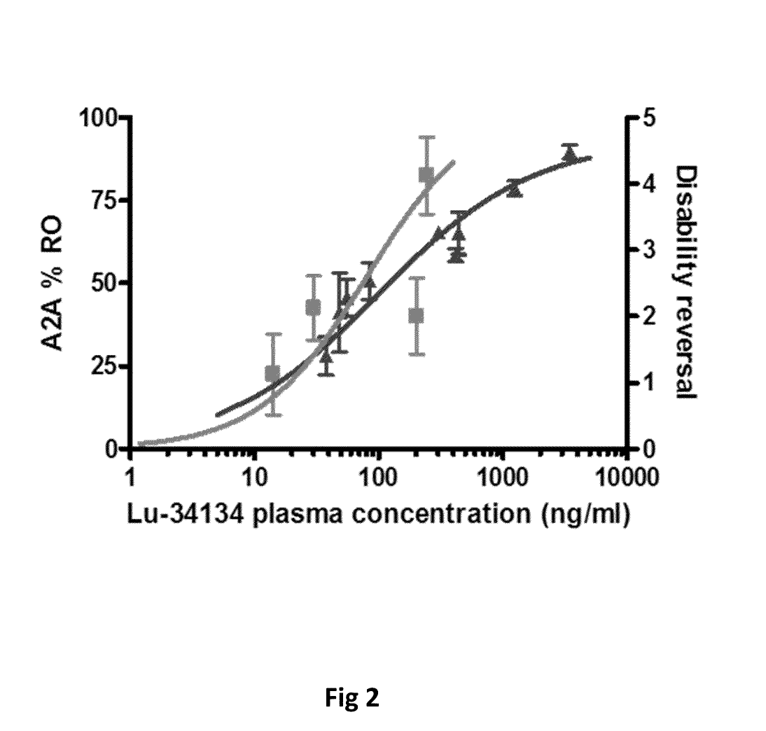

By assuming this minimum A_{2A} occupancy of 80% for clinically relevant efficacy, PK/PD studies were conducted in rats and marmosets. An EC₈₀ plasma concentration of 1250 ng/mL was predicted from the rat occupancy Eₘₐₓ PK/PD model as shown in FIG. 1, Example 1. In marmosets a corresponding 175 ng/mL EC₈₀ plasma concentration was predicted from the Eₘₐₓ PK/PD modelling (FIG. 2, Example 2). Based on these values a human dosage range could be predicted by assuming an oral clearance of 0.6-0.8 L/h and an equal blood:brain ratios in humans, rats and marmosets. Using this this model it was predicted that the daily dose could not be lower than 3 mg and could be as high as 24 mg.

A 10 mg dosage was believed clinical relevant based on the PK/PD modelling because it was assumed high enough to have clinical effects without being too high to induce any of the caffeine-like side effects observed with this class of compounds.

In a first safety and tolerability study the 10 mg dosage was therefore tested. The study was a randomized, double-blind, placebo-controlled, single-ascending study carried out in a total of 17 healthy young men (18-45 years), 19 healthy elderly men (55-75 years), and 14 healthy elderly women (55-75 years) who were administered single doses (1 mg, 5 mg or 10 mg), and 12 subjects were administered single doses 5 mg on three separate occasions (Example 3). The 14C-labelled compound was included in the 10 mg dose in the young men and food effect was also tested at 10 mg. Results from this study showed that the 10 mg dosage was the maximal tolerated dosage because many patients suffered from insomnia and even at the 5 mg dosage these side effects occurred.

This first study in humans showed that both the 10 mg and 5 mg dosage were clinical effective and thus supported the PK/PD occupancy date. However, the clinical dosage gave rise to side effects. Going below 5 mg, on the other hand, gave concerns as to the obtainable clinical effect, as well as receptor occupancy.

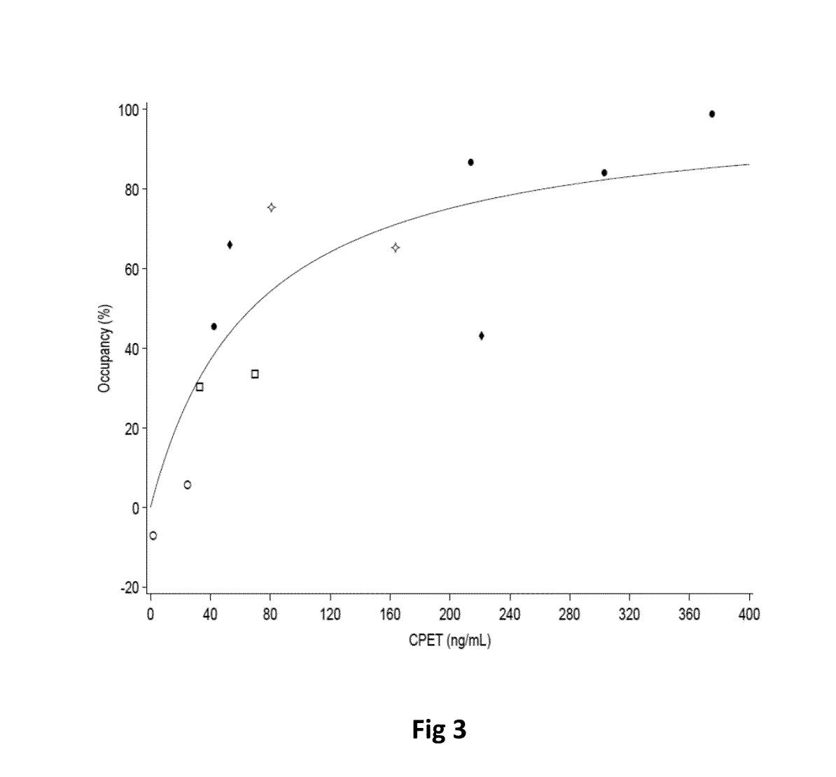

As the clinical relevant dosage seemed to be lower than what could be assumed by looking at the PK/PD data from rats and marmosets, the inventors decided to conduct a positron emission tomography (PET) study in humans to investigate the human occupancy of compound 504 (FIG. 3, Example 4). This study was an interventional, open-label, PET study investigating the occupancy at the A_{2A} receptor after single oral doses in healthy young men using the tracer [11C]-SCH442416. A total of 6 healthy young men were dosed with single oral doses of 0.5 mg to 5 mg. Surprisingly, the inventors found that a clinical effective daily dose with a minimal risk of adverse events seemed feasible with a dosage as low as 3 mg or even 2 mg in humans (FIG. 3, Example 4). This is highly beneficial for the treated subjects because a lower dosage will minimize the adverse events observed with compound 504 at higher dosages, in particular insomnia and sleep phase rhythm disturbance, but also the other caffeine-like signs and symptoms that relates to this class of compounds including dizziness, fatigue, headache, hyperhidrosis, orthostatic hypotension, and palpitations that was observed with the 5 mg and 10 mg dosages (Example 3).

Accordingly, the invention relates to the use of compound 504 for use as a medicament at a daily dosage below or at 3 mg, such as between 0.5 mg and 3 mg, 1 mg and 3 mg, 2 mg and 3 mg, about 2 mg or 2 mg. In particular, the compound may be used for treating PD.

In a further aspect the compound may be used in a pharmaceutical composition or for example a tablet comprising a daily dosage below or at 3 mg, such as between 0.5 mg and 3 mg, 1 mg and 3 mg, 2 mg and 3 mg or about 2 mg or 2 mg.

A further purpose of the present invention is to provide compound 504 for treatment in ADHD (Example 5). Compound 504 was shown to be a potent, orally active A_{2A} receptor antagonist which can elicit a robust in-vivo reversal of a CGS21680-induced hypolocomotion. Across a likely dosage exposure range compound 504 also improved aspects of attentional performance consistent with antagonism of A_{2A} receptors. As such these effects support a potential for compound 504 in the treatment of attentional deficits. Accordingly compound 504 may be used for treating ADHD. The daily dosage may be below or at 3 mg, such as between 0.5 mg and 3 mg, 1 mg and 3 mg, 2 mg and 3 mg or 2 mg.

The mean half-life of compound 504 was similar across dose levels, ranging from 19 to 25 hours. Compound 504 is therefore very distinct to other drugs on the market for treating e.g. ADHD in that it can be given once daily and thus works when the subject wakes up in the morning 24 hours after having taken the medication. Most other drugs in ADHD must be given more regularly since they only work for 2-12 hours for Methylphenidat, 8-14 hours in case of Lisdexamfetamin or in comparison to the other A_{2A} antagonist V-81444 that is given twice daily due to half-life of 5-7 hours.

It is desirable to select a route of administration that is most effective for the therapy, examples thereof being oral administration by use of e.g. tablets.

Preparations suitable for oral administration such as tablets can be produced using, for example, excipients (e.g., lactose and mannitol), disintegrators (e.g., starch), lubricants (e.g., magnesium stearate), binders (e.g., hydroxypropyl cellulose), surfactants (e.g., fatty acid esters) and plasticizers (e.g., glycerin).

The parenteral preparations may also comprise one or more auxiliary components selected from the excipients, disintegrators, lubricants, binders, surfactants and plasticizers described in the above description of oral preparations and diluents, antiseptics, flavors, etc.

In the case of the above-described applications, in general, compound 504 or pharmaceutically acceptable salts thereof may be administered orally or parentally.

In the present context, the term “treatment” and “treating” means the management and care of a patient for the purpose of combating a disease. The term is intended to include the full spectrum of treatments for a given disease from which the patient is suffering, such as administration of the active compound to alleviate the symptoms or complications, to delay the progression of the disease, to alleviate or relief the symptoms and complications, and/or to cure or eliminate the disease. The patient to be treated is preferably a mammal, in particular a human being. In the present context, “disease” can be used synonymous with disorder, condition, malfunction, dysfunction and the like.

### EXAMPLES

### Example 1

Striatal A_{2A} Occupancy in Rats was Investigated by Ex Vivo Autoradiography

The study investigated the receptor occupancy in the striatum after a single oral administration of compound 504 to male rats (Male CD rats (8 weeks old, Charles River Japan). Tritium labelled SCH58561 (0.3 nmol/L from GE Healthcare) was used as tracer for imaging the A_{2A} receptor. The receptor occupancy was determined by incubation of the tracer with brain slices obtained from rats dosed with compound 504.

Compound 504 (0.06 mg/mL) was administered to rats orally at a volume of 5 mL/kg (0.3 mg/kg) and anesthetized 6 and 8 hours after administration with diethyl ether. After that the artery and vein blood was collected into tubes containing heparin Immediately after that the brain was removed and ebbed in OCT compound and frozen in 2-methyl butane cooled with dry ice. The frozen brains were preserved at −80 C until use.

Coronal sections (20 μm) at about bregma 1.2 mm were prepared using a cyromicrotome (Coldtome, set temperature −20 C), Sakura) and placed on a slide glass (Super Frost, Matsunami Glass; 2 or 3 sections per glass). The brain sections collected on the slide glass were preserved at −80 C. Seventy μL of the reaction solution containing the tracer at 0.1 or 0.3 nmol/L was applied on the brain section and incubated at room temperature for the preset time. The reaction solution containing the tracer was handled using siliconized tools. After the incubation with the tracer, the brain section was washed for 1 minute with ice-cold wash solution three times and rinsed with ice-cold water twice. Non-specific binding of the tracer was evaluated by performing the same reaction with the brain section prepared from drug-untreated rats in the presence of 0.1 mmol/l NECA. The brain section was dried at room temperature, exposed to the imaging plate (BAS-TR2040, Fuji Film) in a cassette, and left in a shield box for about 24 hours. The radioactivity was recorded under the following conditions: Resolution: 50 μm; Gradation: 256; Sensitivity: 10000; Latitude:5.

Calibration curves for plasma samples was made by diluting compound 504 with acetonitrile to prepare solutions containing 0.01, 0.03, 0.1, 1, 10 and 30 μg/mL of compound 504. The solutions were added to control plasma samples at an acetonitrile concentration of 1% vol to prepare plasma samples containing compound 504 at 0.1, 0.3, 1, 10, 100 and 300 ng/mL. These plasma samples were used as calibration curve samples.

To pre-treat plasma samples, the internal standard (I.S.) was dissolved in acetonitrile to prepare an I.S. solution at 0.03 μg/mL. Plasma sample was diluted with control plasma whenever necessary. To 0.1 mL of plasma sample, 10 μL of the I.S. solution and 1 mL of 10 mmol/L ammonium acetate were added and stirred to prepare the sample for pretreatment. The sample was pretreated using Oasis HLB Extraction Plate (30 mg, Waters). Each well was serially conditioned with 1 mL of methanol and water. One ml of the sample for pretreatment was loaded on a well. After each well was washed with 1 mL of 5% vol % methanol, the analyte was eluted with 1 mL acethonitrile. The eluate was evaporated under nitrogen gas stream and the residue was redssolved in 0.1 mL of the reconstituted solution. The sample was centrifuged at 4C, about 1800 xG for 5 minutes using UNFILTER (0.45 μm hydrophilic PVDF, Whatman). An aliquote of the filtrate was used for analysis.

Calculation of the A_{2A} receptor occupancy in striatum was done using the below formula

φ(%)=(1−(PSL_{KW}−BG/A)/(PSL_{Ctrl}−BG/A))*100

Where PSL_{KW}- and PSL_{Ctrl} are PSL values in the ROI of compound 504-untreated and treated animal, respectively. PLS is Photo stimulated luminescence for detection of tracer and ROI is region of interest for tracer measurement in striatum.

As the receptor occupancy in each animal, the mean value of 2 or 3 sections (4-6 ROI) was used.

The relationship between the concentration of compound 504 in plasma and captor occupancy is shown in FIG. 1. The EC50 was 121 ng/mL (95% confidence interval, 113 to 128 ng/mL).

### Example 2

A_{2A} Occupancy in MPTP-Lesion Marmosets

The PK/PD relationship of compound 504 was evaluated using the disability reversal score in the more clinically relevant MPTP (1-methyl-4-phenyl-1,2,3,6-tetrahydropyridine)-lesion marmosets mode with the expectation 80% human striatal A_{2A} occupancy is needed for Parkinson treatment (Rose et al (2006) European J Pharm, 546, 82-87; Uchida et al (2014) J Pharmacol Sci, 124, 480-485; Kanda et al (200) Exp Neurol, 162, 321-327).

Marmosets received several MPTP regimens (single MPTP regimen: 2 mg/kg, s.c., per day for 3 consecutive days) before the marmosets were used for PK/PD measurements.

First, rat plasma concentration versus receptor occupancy relationship was established with a PK/PD modelling (E. Effect Model) of temporal striatal A2A occupancy (at 0.3 mg/kg) based on ex vivo slice autoradiography after evaluating for potential hysteresis using a semi compartment PK model (equilibration half-life<0.5 h). The EC₈₀ of the rat striatal occupancy was predicted to be 1250 ng/mL from the PK/PD modelling (see also FIG. 1).

Second, PK/PD modelling (Eₘₐₓ Effect Model) of plasma concentration versus disability reversal score of the MPTP-treated marmosets (0.1-3 mg/kg) was established with a predicted EC₈₀ of 175 ng/mL which corresponds to >50% A_{2A} occupancy extrapolated from the fitted rat occupancy curve. In the absence of any striatal occupancy data in marmoset to relate plasma concentration needed for EC₈₀ A_{2A} occupancy for human dose projection, a daily dose range of 3-24 mg was established based on predicted EC₅₀₋₈₀ plasma concentrations of rat striatal occupancy and marmoset disability reversal score.

In FIG. 2 the predicted therapeutically effective EC₅₀₋₈₀ compound 504 plasma concentration range in PD patients based on rat A_{2A} occupancy and MPTP-treated marmoset disability reversal scores is shown.

### Example 3

Safety and Tolerability Study

The main objective of the study was to investigate the safety and tolerability of Compound 504 given as single doses to healthy young men aged ≧18 and ≦45 years and elderly men and women aged ≧55 and ≦75 years with a body mass index (BMI)≧19 and ≦29 kg/m2. The study was conducted in three parts: Parts A, B, and C.

Parts A and B were single-dose escalations to determine the safety and tolerability in young healthy men, and in elderly men and women, respectively. Three doses (Cohorts A1 to A3) were tested in healthy young men: 1 mg (N=6), 5 mg (N=6), and 10 mg (N=5); placebo (N=8, total of the three cohorts). 14C-labelled compound 504 (250 nCi) was included in the 10 mg dose given to healthy young men. Two doses (Cohorts B1 and B2) were tested in healthy elderly men and women: 5 mg (N=9 men/4 women), 10 mg (N=4 men/4 women); placebo (N=6 men/4 women, total of the three cohorts). In Part C, repeated single doses were used to investigate the possible effect of food and the intra-subject variability following administration of compound 504. A total of 12 healthy elderly men (N=6) and women (N=6) each received a single dose of compound 504 5 mg on three separate occasions in a randomised manner. On one occasion, compound 504 was administered after an FDA standard high-fat breakfast, and on two occasions, Compound 504 was administered after an overnight fast. A washout period of at least 7 days separated each dose administration. In Parts A and B, compound 504 was administered as an oral solution (0.25 mg/mL) and in Part C, compound 504 was administered as a solid formulation (5 mg capsules).

The pharmacokinetic parameters of compound 504 determined in Parts A, B, and C of the study is summarized below

Single-dose Pharmacokinetic Parameters of
Compound 504 in Healthy Young Men -Part A

| | 1 mg | | 5 mg | | 10 mg | |
|---|---|---|---|---|---|---|
| | Compound 504 | | Compound 504 | | Compound 504 | |
| | N = 6 | | N = 6 | | N = 5 | |
| AUC_{0-inf} | 2661 | (56.5) | 12589 | (35.3) | 18102 | (19.2) |
| (h·ng/mL) | | | | | | |
| Cₘₐₓ | 82.7 | (21.5) | 423 | (12.9) | 787 | (13.5) |
| (ng/mL) | | | | | | |
| tₘₐₓ (h) | 1.25 | (0.50, 2.50) | 1.75 | (1.00, 4.00) | 1.00 | (0.50, 2.50) |
| t_{1/2} (h) | 24.3 | (66.8) | 25.0 | (26.0) | 19.3 | (26.6) |
| CL/F (L/h) | 0.541 | (72.7) | 0.440 | (35.3) | 0.569 | (18.9) |
| V_{z}/F (L) | 13.4 | (23.0) | 14.8 | (14.7) | 15.3 | (18.7) |

Arithmetic mean (CV %) values are presented.
Median (min, max) values are presented for tₘₐₓ.
N = number of subjects

Single-dose Pharmacokinetic Parameters of Compound 504 in Healthy Elderly Men and Women - Part B

| | 5 mg Compound 504 | | | | | | 10 mg Compound 504 | | | | | |
|---|---|---|---|---|---|---|---|---|---|---|---|---|
| | Men | | Women | | Overall | | Men | | Women | | Overall | |
| | N = 9 | | N = 3 | | N = 12 | | N = 4 | | N = 4 | | N = 8 | |
| AUC_{o-inf} | 9587 | (37.2) | 9548 | (28.9) | 9577 | (34.1) | 15660 | (55.9) | 14069 | (29.2) | 14865 | (43.0) |
| (h·ng/mL) | | | | | | | | | | | | |
| Cₘₐₓ | 356 | (24.5) | 485 | (26.2) | 388 | (28.1) | 652 | (12.8) | 781 | (4.45) | 717 | (12.7) |
| (ng/mL) | | | | | | | | | | | | |
| tₘₐₓ | 1.00 | (0.50, 3.00) | 1.00 | (1.00, 2.50) | 1.00 | (0.50, 3.00) | 0.750 | (0.50, 1.00) | 0.750 | (0.50, 1.00) | 0.750 | (0.50, 1.00) |
| (h) | | | | | | | | | | | | |
| t_{1/2} | 23.5 | (37.6) | 17.4 | (17.1) | 22.0 | (37.0) | 21.6 | (46.3) | 18.8 | (25.4) | 20.2 | (36.7) |
| (h) | | | | | | | | | | | | |
| CL/F | 0.611 | (47.3) | 0.556 | (30.5) | 0.597 | (43.2) | 0.908 | (78.6) | 0.780 | (40.6) | 0.844 | (61.1) |
| (L/h) | | | | | | | | | | | | |
| V_{z}/F | 18.0 | (20.0) | 13.6 | (18.1) | 16.9 | (22.5) | 21.6 | (22.5) | 19.6 | (9.40) | 20.6 | (17.3) |
| (L) | | | | | | | | | | | | |

Arithmetic mean (CV %) values are presented.
Median (min, max) values are presented for tₘₐₓ.
N = number of subjects

Statistical Analysis of the Effect of Food on the Pharmacokinetic
Parameters of Compound 504 in Elderly Men and Women -Part C

| | 5 mg Compound 504 | | |
|---|---|---|---|
| | Fed | Fasting | Ratio of LS |
| | (N = 12) | (N = 24) | Means (90% CI) |
| AUC_{0-inf} | 5961^{a} | 6311 | 0.945 (0.839, 1.064) |
| (h·ng/mL) | | | |
| AUC₀₋ₜ | 5558 | 5961 | 0.932 (0.840, 1.036) |
| (h·ng/mL) | | | |
| Cₘₐₓ | 269 | 348 | 0.772 (0.726, 0.821) |
| (ng/mL) | | | |
| t_{1/2} (h) | 15.8^{a} | 14.9 | 1.059 (0.934, 1.202) |

^{a}N = 11
CI = confidence interval;
LS = least squares;
N = number of subjects

The results of Parts A and B showed that compound 504 was rapidly absorbed at each dose, with a median tₘₐₓ values of 0.75 to 1.75 hours post-dose. After reaching Cₘₐₓ, the plasma concentration of Compound 504 plateaued until approximately 5 hours post-dose; thereafter, the mean plasma concentrations declined, although individual plasma concentrations fluctuated. Exposure to Compound 504, as determined by AUC₀₋₂₄ and Cₘₐₓ, appeared to increase in an approximate dose-proportional manner in the dose range 1 to 10 mg. The mean t_{1/2} was similar across doses and ranged from 19 to 25 hours.

In Part B, there were no marked differences in exposure to Compound 504 between men and women. The mean t_{1/2} was generally similar between sexes and between the 5 and 10 mg doses.

There were no apparent differences in the pharmacokinetic profile of Compound 504 between healthy young subjects and elderly subjects.

In Part C, food delayed the absorption of Compound 504; the median tₘₐₓ occurred approximately 2 to 3 hours later in the fed state. The mean Cₘₐₓ was approximately 23% lower in the fed state than in the fasting state. Overall exposure, based on AUC_{0-inf} and AUC₀₋ₜ, was not statistically significantly different following administration of Compound 504 5 mg in the fed state compared to the fasting state. In each dietary state, overall exposure to Compound 504 (based upon examination of mean AUC) appeared to be higher in men, than in women. For men, the intra-subject variability (CV %) across the fasting doses was low, 27% and 16% for AUC0-inf and Cₘₐₓ, respectively. For women, variability was higher, with CV % of 59% and 23% for AUC_{0-inf} and Cₘₐₓ, respectively.

In Part A, single doses of 1, 5, and 10 mg of compound 504 were administered to healthy young men.

In total, 13 of the 17 subjects in the compound 504 dose groups had 36 AEs and 2 of the 8 subjects in the placebo group had 3 AEs (adverse events).

Insomnia was the most common adverse event, reported in 4 subjects in the compound 504 5 mg group and by all 5 subjects in the compound 504 10 mg group. Insomnia was not reported in subjects in the placebo or compound 504 1 mg groups. Insomnia was generally reported approximately 14 to 16 hours post-dose, which corresponded to a clock time of approximately 23:00 to 01:00. Eight events of insomnia were mild and one was moderate. Insomnia generally resolved within 2.5 to 6.25 hours; in 2 subjects, insomnia lasted for approximately 2 days.

In Part B, single doses of 5 mg and 10 mg of compound 504 were administered to healthy elderly men and women.

In total, 16 of the 21 subjects in the compound 504 dose groups had 53 AEs and 5 of the 10 subjects in the placebo group had 7 AEs.

Three subjects had 5 severe adverse events in the compound 504 10 mg group: 1 woman had insomnia, affect lability, and restlessness; 1 man had blood pressure increased; 1 woman had ventricular extrasystoles. The ventricular extrasystoles occurred during telemetry (approximately 3 hours post-dose), and the extrasystoles were seen intermittently up to 1 day 4 hours post-dose. The subject was, after completion of the study, referred to an independent external cardiologist who examined the subject, and similar events were detected on Holter recordings 51 days after dosing.

The AEs with the highest incidences in the compound 504 dose groups in Part B were insomnia and restlessness, which was consistent with the findings in Part A of the study. The 10 mg dose was considered to approach the maximum tolerated dose in elderly subjects.

In Part C, single doses of 5 mg of compound 504 were administered to healthy elderly subjects were safe and tolerated when given in the fed and the fasting states.

In total, 11 of the 12 subjects had 90 AEs, of which 15 events were moderate and 3 events were severe. The 3 severe events occurred in lsubject: restlessness, affect liability, and disturbance in attention, which occurred 30 minutes, 1 hour, and 1 hour post-dose following the third single dose of Compound 504 (5 mg, fasting), respectively. The events resolved without treatment after 1.5 days and during this time the subject also had moderate anxiety and mild insomnia.

The AE with the highest incidence was insomnia and there were 12 events of mild insomnia and 2 events of moderate insomnia. This finding was consistent with the findings in Parts A and B of the study.

### Example 4

Pet Study in Humans Investigating the A_{2A} Receptor Occupancy

The study was designed as an open-label, positron emission tomography (PET) study investigating A_{2A} receptor occupancy after single oral dosing of compound 504 in young healthy men using [11C]-SCH442416 as tracer compound.

The study consisted of a Screening Period, a Magnetic Resonance Imaging (MRI) scan, a Safety Baseline, a Treatment Period (a Baseline PET scan, compound 504 dosing, and two treatment PET scans), and a Follow-up.

There were 3 cohorts in the study, each including 2 subjects. Two subjects in Cohort A1 received 5 mg compound 504; in Cohort A2, 1 subject received 0.5 mg compound 504 and 1 subject received 1 mg compound 504; in Cohort A3,1 subject received 2 mg compound 504 and 1 subject received 3 mg compound 504.

The number of subjects, dose of compound 504, and the assessment time points were reviewed and adjusted based upon preliminary pharmacokinetic, pharmacodynamic, and safety and tolerability data from the previous cohorts.

In the Screening period (Days −28 to −4) safety screening procedures were performed and subject eligibility was confirmed following a magnetic resonance imaging (MRI) scan (Days −21 to −2). Eligible subjects were admitted to the clinic on Day −1. On Day 1, a baseline PET scan was performed prior to receiving a single dose of compound 504. A second PET scan (PET2) was performed at approximately 2 hours post-dose, which was the estimated time of maximum compound 504 plasma concentration (tₘₐₓ). The subjects in Cohort A1 had a third PET scan (PET3) approximately 24 hours post-dose and the subjects in Cohorts A2 and A3 had a third PET scan (PET3) approximately 26 hours post-dose. The subjects received the PET tracer [11C]-SCH442416 immediately prior to each PET scan.

All subjects were confined to the clinic from Day −1 until completion of the 72 hour post-dose safety assessments on Day 4. Follow-up assessments were performed on Day 6 (up to 1 day before and 2 days after). For each subject the total duration of the study, from screening to follow-up, was approximately 2.5 to 3.5 weeks.

The PET tracer, [11C]-SCH442416 was provided as a radiopharmaceutical in an 11 mL sterile and pyrogen free type 1 glass vial as a solution for intravenous administration. The [11C]-SCH442416 solution consisted of up to 10% ethanol and 90% saline and was administered as an intravenous bolus in approximately 20 mL of the saline/ethanol solution over approximately 20 seconds. Each subject received a maximum of 500 MBq of [11C]-SCH442416 for each PET scan.

At predetermined time points, blood samples were drawn for drug concentration analysis of compound 504 and safety and tolerability were assessed.

Main Inclusion Criteria

Men between 25 and 55 years of age (extremes included), with a body mass index (BMI) between 18.5 and 30 kg/m² (extremes included)

Pharmacokinetic Assessments

The following pharmacokinetic parameters of compound 504 were calculated for each subject: area under the plasma concentration-time curve from zero to infinity (AUC_{0-inf}); area under the plasma concentration-time curve from zero to time Hast (AUC₀₋ₜ); maximum observed plasma concentration (Cₘₐₓ); plasma concentration at the time of PET scanning calculated for each post dose PET scan (C_{PET}); oral clearance (CL/F); apparent elimination half-life in plasma (t_{1/2}); nominal time corresponding to the occurrence of Cₘₐₓ (tₘₐₓ) time of last quantifiable concentration (tlₐₛₜ) and apparent volume of distribution (Vz/F).

Pharmacodynamic Assessments

PET data were analysed using the simplified reference tissue method (SRTM). This method assumed a reference region (in this instance the cerebellum) that is similar to the target-rich regions, except that it is devoid of the target receptor. The SRTM implementation employed directly estimated the binding potential relative to the non-displaceable component (BPND), which can be thought of as a measure of specific binding.

Receptor occupancy (RO) in a region of interest (ROI) can then be calculated directly as: RO=100×(1−BPND(post-dose)/BPND(baseline))

The dorsal putamen and globus pallidus were the two ROIs with the highest specific signal and thus RO was calculated for these ROIs.

Statistical Methodology

The following analysis sets were used:

all-subjects-treated set (ASTS)—all subjects who were administered compound 504 and hence [11C]-SCH442416

pharmacodynamics set (PDS)—all subjects who had at least one valid post-IMP PET scan and a corresponding valid assessment of CPET

The pharmacokinetic parameters of compound 504 were estimated using non-compartmental analysis.

Binding potential and estimated RO were listed and presented graphically versus CPET.

The relationship over time between plasma concentrations of compound 504 and the estimated A_{2A} receptor occupancy was analysed by means of regression by applying an Eₘₐₓ model and investigated.

Results

FIG. 3 shows the receptor occupancy in the putamen versus the compound 504 plasma concentration at the time of the PET scan estimated using the Eₘₐₓ.

Compound 504 was rapidly absorbed with a tₘₐₓ of 1 to 2 hours, which corresponded to the time of the first post-dose PET scan (PET2). The estimated Eₘₐₓ and EC₅₀ values were 88% and 31.0 ng/mL in the putamen and 101% and 68.9 ng/mL in the globus pallidus. The Eₘₐₓ model generally appeared to be a good fit for the putamen data, the variability was higher for the globus pallidus data; the residual plots for both sets of analysis showed no evidence to suggest the assumptions of the model were invalid.

From these data it's evident that dosage at or below 3 mg is sufficient to reach an occupancy of 80%.

### Example 5

The purpose of this study was to investigate compound 504 on the performance of Long Evans rats in a test of attention, the 5-choice serial reaction time task (5-CSRTT; Robbins, Psychopharmacology (Berl) (2002) 163(3-4):362-80.).

The study consisted of 3 phases.

(1) An investigation of compound 504 against the hypolocomotion induced by the selective A_{2A} receptor agonist CGS21680 (Higgins et al Behav Brain Res (2007); 185 (1): 32-42). This test serves as a pharmacodynamic assay to establish doses of compound 504 necessary to functionally block a centrally mediated A_{2A} receptor mediated response. Plasma levels of compound 504 were also measured at timepoints equivalent to the behavioural test to determine plasma exposure at pharmacologically relevant doses of drug.

(2) Based on doses established from (1), compound 504 was tested in 4 variants of the 5-CSRTT designed to measure effect of drug on distinct aspects of performance. (A) Testing under standard conditions, i.e. equivalent to final conditions to which the animals were trained, i.e. stimulus duration (SD) 0.75 seconds (s), inter-trial interval (ITT) 5 s, 100 trials. The purpose of this condition being to measure drug effect on performance under standard conditions. (B) Testing under conditions of extended (long) ITI, i.e. low event rate, SD 0.3 s, ITI 5, 7.5, 10 s, 120 trials. The purpose of this condition being to measure drug effect on performance under lower event rate requiring the animal to delay its responding. (C) Testing under conditions of short ITI, i.e. high event rate, SD 0.3 s, ITI 3, 4, 5 s, 120 trials. The purpose of this condition to measure drug effect on performance under high event rate requiring the animal to rapidly process sensory information. (D) Testing under conditions of extended trials, i.e. SD 0.3 s, ITI 5 s, 250 trials. The purpose of this condition to measure drug effect on performance under extended trials, testing sustained attention or vigilance.

(3) An investigation of compound 504 on baseline locomotor activity in rats well habituated to the test apparatus.

Doses of 0.01, 0.03, 0.06, 0.1 mg/kg were tested in a dosage form of 0.5 w/v % methyl cellulose 400 (viscosity: 400 cP) in distilled water. Drug was administered at a volume of 5 ml/kg, oral.

Phase 1:

Sixty (60) male Long Evans rats, 3 months age, were allocated into 6 groups of N=10 rats per group. The 6 groups are listed in the table below:

TABLE 1
Summary of Treatments for Phase 1

| | | Number of |
|---|---|---|
| Group | Test Article | Animals |
| 1 | Vehicle/Vehicle | 10 |
| 2 | Vehicle/CGS21680 1 mg/kg | 10 |
| 3 | compound 504 0.01 mg/kg/CGS21680 1mg/kg | 10 |
| 4 | compound 504 0.03 mg/kg/CGS21680 1mg/kg | 10 |
| 5 | compound 504 0.06 mg/kg/CGS21680 1mg/kg | 10 |
| 6 | compound 504 0.1 mg/kg/CGS21680 1mg/kg | 10 |

Compound 504 was administered by oral gavage 60 minutes prior to activity testing. CGS21680 was administered subcutaneously 10 minutes prior to activity testing. The activity test, monitoring the rat's spontaneous activity was measured in the automated Med Associates activity test chamber for 20 minutes. The tracking arena was of dimension 17″W×17″L×12″H, sensor bars were secured 1″ above the floor to track distance travelled, and a second set of sensor bars were placed 6″ above the floor to measure vertical movement and rearing activity. The parameters set on the tracking software were: resolution—50 ms, box size—4 beams, resting delay—500 ms, and ambulatory trigger—2.

Following a washout period of one week, 20 rats used in the activity study were reallocated to 4 new groups of n=5 per group, and each treated with compound 504 at the doses used in the activity study (0.01, 0.03, 0.06, and 0.1 mg/kg). A minimum of 400 uL of whole blood was collected via saphenous bleed at 10 min, 20 min, 60 min, and 120 min post drug administration, i.e. at time points that coincided with the activity study (and phase 2: 5-choice experiments). Blood was collected and transferred into K3 EDTA tubes. The tubes were allowed to sit at room temperature for approximately 2 minutes and placed on wet ice until centrifugation. Blood was spun in the centrifuge with speed of 3,500 rpm for 15 minutes at 4 oC. Plasma was separated and placed into a labeled 0.75 ml Matrix tube and capped. Plasma tubes were placed into a Matrix rack and stored in a −80 oC freezer. At the final time point (120 minutes post drug administration) following blood collection by cardiac puncture, the brain tissue was extracted and hemisected along the midline. One hemisphere, without the cerebellum, was placed in the 15 ml pre-weighed brain tube while the other portion was disposed. The brain tubes were weighed, frozen on dry ice, and stored at −80 C. Brain tissue and plasma samples were shipped on dry ice to Sponsor. Blood and brain levels of compound 504 were to be conducted by the Sponsor.

Phase 2:

Sixteen (16) experimentally naïve, male Long Evans rats were trained to asymptotic performance in the 5-CSRTT over a period of approximately 2-3 months. The rats were trained to final stimulus duration (SD) of 0.75 s, 5 s inter-trial interval (ITI), 5 s limited hold (LH), 100 trials per session. Target performance levels under these conditions was in the range of >80% accuracy and <20% omissions. Drug testing with compound 504 began once performance was stable.

Once the group of animals were trained, multiple drug studies according to various experimental tasks (see table 2 below) was tested in the same cohort of rats. Doses of compound 504 were established based on outcomes from Phase 1. The study design was made up of 4 experiments, with each experiment 2-3 weeks in duration and a 1 week intervening period between each experiment.

TABLE 2
Summary of 5-CSRTT schedules for Phase 2

| | | Time- |
|---|---|---|
| Exp. | Task | line |
| 1 | Examination of compound 504 at 0.01, 0.03, 0.06, | 3 weeks |
| | 0.1 mg/kg + vehicle control (i.e. 5 treatment cycles). | |
| | Test under standard conditions. | |
| 2 | Examination of compound 504 at 0.03, 0.06 mg/kg + | 3 weeks |
| | vehicle control (i.e. 3 treatment cycles). | |
| | Test under conditions of multiple Inter-trial-interval (ITI) | |
| 3 | Examination of compound 504 at 0.03, 0.06 mg/kg + | 3 weeks |
| | vehicle control (i.e. 3 treatment cycles). | |
| | Test under conditions of short multiple ITI | |
| 4 | Examination of compound 504 at 0.03, 0.06, | 3 weeks |
| | 0.1 mg/kg + vehicle (i.e. 4 treatment cycles). | |
| | Test under extended 250 trials | |

Phase 3:

Sixteen (16) male Long Evans rats were habituated to Med Associates test activity chambers (17″ W×17″ L×12″ H) over three, 90-minute sessions. The effect of compound 504 and amphetamine (1 mg/kg) was investigated on motor behaviour over a 90 min session using a repeated measures design with a washout period of 2-3 days between each treatment cycle. Total distance travelled and rearing counts for the total session were the primary measure. Compound 504 at 4 doses (0.01, 0.03, 0.06, and 0.1 mg/kg), amphetamine at a single dose (1 mg/kg) plus a vehicle control was tested.

Results

A_{2A} receptor agonist CGS21680.

Pretreatment with CGS21680 (available from e.g. Merck Millipore Corporation) (1 mg/kg SC) produced a robust hypolocomotion in rats pretreated with vehicle. Locomotor activity, defined as total distance travelled, and rearing, both measured over the 20 min test period were reduced by 92% and 99% respectively compared to vehicle treated controls. Compound 504 (0.01-0.1 mg/kg) adminstered orally 50 min before CGS21680 produced a dose-related antagonism of the CGS-hypolocomotion with an approximate (uncalculated) ED50 of 0.06 mg/kg. At a higher dose of 0.1 mg/kg, compound 504 completely reversed the CGS-induced hypolocomotion. Similar effects of compound 504 were noted on measures of rearing and ambulatory episodes. Formal analyses revealed main effects of treatment on distance travelled (F5.56=33.9, P<0.01), rearing (F5.56=35.1, P<0.01) and ambulatory episodes (F5.56=37.7, P<0.01). In each case, compound 504 significantly reversed the CGS-hypolocomotion at the 0.06 and 0.1 mg/kg doses, with the 0.1 mg/kg dose at the vehicle baseline.

Although the bioanalytical measures from animals pretreated with compound 504 were not conducted by IVS, these data are included in the present report for comparison to the behavioural measures. Compound 504 demonstrated an orderly dose-plasma exposure relationship over the dose range 0.01-0.1 mg/kg. An almost linear relationship between reversal of CGS-hypolocomotion and plasma [drug] was evident at both the 60 min and 90 min sampling periods which corresponded to the time of testing. Plasma levels of range 150-400 ng/ml were attained over the pharmacologically active dose range of 0.03-0.1 mg/kg compound 504 at these timepoints.

Phase 2: Effect of compound 504 on attentional performance as measured by the 5-choice serial reaction time task.

Over a period of approximately 10 weeks, 16 male Long Evans rats were trained to the 5-CSRTT. Out of this group, 15 reliably met the performance criterion of >80% correct responses and <20% missed trials under the final baseline conditions of 0.75 s SD, 5 s ITI, 100 trials, 5 s LH. Thus for the majority of studies, the sample size was 15 with all rats receiving each treatment in a counterbalanced sequence.

Standard conditions: compound 504 (0.01-1 mg/kg oral) was first evaluated on 5-CSRTT performance under the standard baseline conditions. A total of 15 rats were used in this study cohort. Main effects of treatment were noted for correct latency (F4.56=3.26, P=0.02) and premature responses (F4.56=3.53, P=0.01). Compound 504 significantly increased speed of responding (i.e. reduced correct latency) at doses 0.03-0.1 mg/kg, and increased premature responding, although this only reached significance at the 0.1 mg/kg dose. All other measures such as % correct, # correct/incorrect responses, omissions, magazine latency was unaffected by compound 504 at the doses tested.

Long ITI: Next, compound 504 (0.03-0.06 mg/kg oral) was tested under conditions of long ITI. The cohort size was 15. Extending the ITI had a significant effect on premature responses (F2.28=93.3, P<0.01), and also the number of correct/incorrect trials and omissions (F2.28>7.9, P<0.01), although % correct and correct latency was unaffected. There was no main effect of compound 504 or Lu×ITI interaction on any measure except on premature responses (F2.28=4.25, P=0.02) which were increased by compound 504 (0.06 mg/kg). A non-significant trend of compound 504 to reduce accuracy specifically under long ITI was evident (F2.28=2.62, P=0.09) at this same dose.

Data from this experiment were also analysed for treatment averaged over the multiple ITI. The only main effect of treatment was premature responses (F2.28=4.24, P=0.02).

Short ITI: Compound 504 (0.03-0.06 mg/kg oral) was tested under conditions of short ITI. The cohort size was 14, (one rat removed (M3) due to inconsistency of performance). Reducing the ITI had a marked effect on premature responses, # correct/incorrect trials, omissions (F2.26>3.42, P<0.05), with reducing the ITI increasing task difficulty. Percent correct was affected by ITI (F2.26=3.61, P=0.04). Main effects of treatment on % correct, incorrect responses, omissions, premature responses were noted (F2.26>4.91, P<0.01) which reflected that compound 504 improved performance under this condition in terms of choice accuracy, particularly at the short (3s) ITI.

Data from this experiment were also analysed for treatment averaged over the multiple ITI. Main effects of treatment were noted on measures of % correct, # incorrect responses, omissions, premature responses (F2.26>4.75, P<0.01) with compound 504 improving performance on each measure, i.e. increasing % correct, reducing incorrect responses and omissions. Correct latency was of borderline significance (F2.26=3.31, P=0.05) reflecting the trend for compound 504 to increase response speed.

Extended 250 trials: compound 504 (0.03-0.1 mg/kg oral) was tested under conditions of extended 250 trials. Because of the importance to evaluating performance over session duration, these data are presented with trials organised into 5 blocks of 50 trials. The cohort size was 13, two rats were removed due to inconsistency of performance

Over the course of these sessions, the vehicle pretreated rats over the first 150 trials (Bins 1-3) performed at a stable level of responding of ˜80% correct, however this accuracy dropped to 52+10% accuracy by Bin 5, with 4/13 rats failing to complete any correct trials during the 5th bin. Correct responses declined from a mean of 25-27 responses in Bins 1-3, to 14.6+3.3 responses in Bin 5. Omissions, i.e. trials initiated but not completed, also increased across the session, with 8.2+1.4 omissions in Bin 1, rising to 19.1+4.4 by Bin 5. It is important to note that this decline in rewards earned is not due to satiety for if the rats are given free access to 250 food pellets over a time period equivalent to the 250 trials schedule, all pellets are consumed. Rather, the decline likely reflects a decline in vigilance or motivation to continue on task.

Pretreatment with compound 504 (0.03-0.1 mg/kg oral) protected against the decline in % correct and number of correct responses, and increase in omissions over Bins 4 and 5—particularly at the 0.06-0.1 mg/kg doses. All rats completed at least 13 correct trials in Bin 5 following 0.1 mg/kg dose of compound 504. Thus a significant treatment×bin interaction was noted for no. correct responses (F12.144=4.58, P<0.01) and omissions (F12.96=3.27, P <0.01), reflecting the improved performance (higher hit rate) at Bins 4 and 5 following compound 504 pretreatment. Total correct trials were also significantly increased in compound 504 (0.1 mg/kg) pretreated rats relative to vehicle control. Percent correct was of borderline significance (treatment x bin interaction: F12.144=1.54, P=0.1).

Phase 3: Effect of compound 504 on locomotor activity in habituated rats: comparison to amphetamine

Compound 504 (0.01-1 mg/kg oral) produced a dose related increase in locomotor activity, measured as distance travelled (F5.70=21.7, P<0.01) and rearing counts (F5.70=17.9, P<0.01) over a 90 min test session. The magnitude of this effect plateaued at the 0.06-0.1 mg/kg dose. The effect size of compound 504 was significantly less that of a single acute dose of amphetamine (1 mg/kg IP) included in the same study.

The timecourse for the locomotor activity changes produced by compound 504 and amphetamine were also evaluated. Compound 504 activity peaked during the first 10 min timebin and declined gradually over the 90 min session. The temporal pattern of effect was similar for all doses of compound 504.

Compound 504 was shown to be a potent, orally active A_{2A} receptor antagonist which can elicit a robust in-vivo reversal of a CGS21680-induced hypolocomotion at doses ranging from 0.01-0.1 mg/kg, corresponding to plasma concentrations of range 100-450 ng/ml. Across this same dose (and likely exposure) range, compound 504 also improved aspects of attentional performance of rats measured in the 5-CSRTT, consistent with antagonism of A_{2A} receptors. Specifically, increased accuracy and number of correct responses under the sITI protocol, i.e. high event rate, and also improving performance over extended trials suggesting improvement in sustained attention/vigilance. As such these effects support a potential for compound 504 in the treatment of ADHD.

1. A method of treating Parkinson's Disease comprising administering a compound of formula 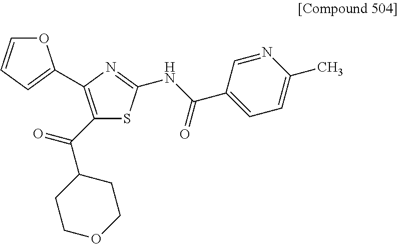 [SMILES: Cc1ccc(C(=O)Nc2nc(-c3ccco3)c(C(=O)C3CCOCC3)s2)cn1]
or a pharmaceutical salt thereof in a dosage of between 2 mg and 3 mg per day to a patient in need thereof.

2. The method according to claim 1, wherein the compound is administered in a once daily dosage.

3. The method of claim 2, wherein the dosage is 2 mg per day.

4. The method of claim 2, wherein the dosage is 3 mg per day.

5. A method of treating ADHD comprising administering a compound of formula 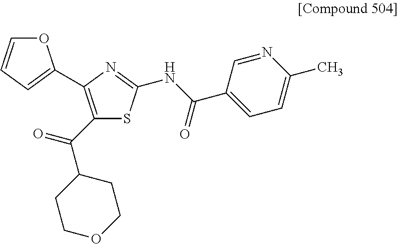 [SMILES: Cc1ccc(C(=O)Nc2nc(-c3ccco3)c(C(=O)C3CCOCC3)s2)cn1]
or a pharmaceutical salt thereof in a dosage of between 2 mg and 3 mg per day to a patient in need thereof.

6. The method of claim 5, wherein the compound is administered in a once daily dosage.

7. The method of claim 6, wherein the dosage is 2 mg per day.

8. The method of claim 6, wherein the dosage is 3 mg per day.

9. A pharmaceutical composition comprising a dosage of between 0.5 mg and 3 mg of a compound of formula 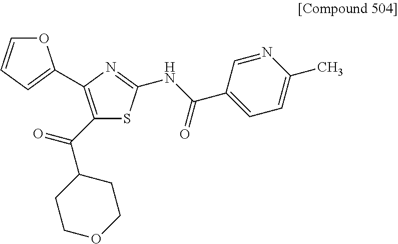 [SMILES: Cc1ccc(C(=O)Nc2nc(-c3ccco3)c(C(=O)C3CCOCC3)s2)cn1]
or a pharmaceutical salt thereof.

10. The pharmaceutical composition of claim 9, wherein the composition comprises 2 mg of the compound.

11. The pharmaceutical composition of claim 9, wherein the composition comprises 3 mg of the compound.

12. The pharmaceutical composition of claim 9, wherein the pharmaceutical composition is a tablet.

13. The pharmaceutical composition of claim 9, wherein the pharmaceutical composition comprises further excipients or diluents.

14. The pharmaceutical composition of claim 12, wherein the tablet comprises further excipients or diluents.
